(19) United States
(12) Patent Application Publication
(10) Pub. No.: US 2010/0170036 A1
(43) Pub. Date: Jul. 8, 2010

(54) DISABLED TRANSFER BOARD

(76) Inventor: ROOZBEH SHIRANDAMI, Farnborough, Kent (GB)

Correspondence Address: Mr. Roozbeh Shirandami, 33 Bassetts Way, Farnborough, Kent, BR6 7AF, GB

(21) Appl. No.: 12/663,832

(22) PCT Filed: Jun. 5, 2008
(86) PCT No.: PCT/GB08/01936

§ 371 Date: Dec. 9, 2009

(30) Foreign Application Priority Data
Jun. 13, 2007 (GB) 0711305.3

(51) Int. Cl. A61G 7/10; A61F 5/37

(52) U.S. Cl. 5/81.1 R; 128/870

(57) ABSTRACT

A transfer board with at least a pair of elongated elements interconnected by means of at least one hinge allowing the elongated members to fold on each other when not in use, wherein the said hinge forms a rigid joint between the said elongated members when the said members are unfolded for use; and wherein the location of the hinge is offset to one side of the joint centerline such that the rigid joint formed between the connecting parts is not primarily-dependent on the mating surfaces perpendicular to the long axis of the said elongated members and forming an alternative primary load path between the soffit of the first elongated member and the cooperating upper surface of the projecting connector of the second elongated member. In this respect the gap between the elongated members remains constant under normal use and eliminates a possible pinching point.

This invention relates to a disabled transfer board. A transfer board (or more commonly known as a “transfer board) is used as an aid for transferring the disabled person from one item of furniture/support device to another, either by themselves or by assistance.

The transfer boards currently available are basically a rigid board of fixed length and width, typically 600-700 mm long and 200 mm wide. The board basically forms a bridge between the two items of furniture/support device and allows the disabled person/user to slide from one item of furniture/support device to another. To facilitate sliding of the disabled person one face (i.e. the sliding face) is highly polished while the ends of the opposing face in contact with either furniture/support surfaces may be treated by non-slip paint to prevent the board from slipping against the said furniture/support device.

Such boards although are very useful in aiding the disabled have a major disadvantage of not being easily portable because of their size and in particular their length which range between 60 to 100 cm.

Prior arts EP0233782, U.S. Pat. No. 6,349,432, U.S. Pat. No. 5,966,754, U.S. Pat. No. 5,065,464 and U.S. Pat. No. 4,744,115 indicate transfer boards which include flexible hinge between various segments. These joints however are formed by flexible straps which can not be locked under the application of load to the sliding surface. Subsequently the leaf attached by such joint in all cases are self bridging to avoid the requirement of the joint being a loaded member and therefore rigid.

Shirandami Patent GB2387321 overcomes this drawback by allowing the board to be folded so as to reduce its length when not in use and therefore making it easier to transport. According to the said invention, there is provided a transfer board comprising of at least a pair of elongated elements interconnected by means of at least one hinge allowing the elongated members to fold on each other when not in use, wherein the said hinge connecting the said elongated member forms a rigid joint between the said elongated members under the action of load applied to the sliding surface of the transfer board when the said members are unfolded for use. The joint between the said elongated members however may form a pinching point for the user.

A main aspect of the present invention therefore lies in providing an improved joint between the said elongated members of the type disclosed in UK patent GB2387321 to prevent the aforementioned pinching point. Specifically the improvement takes the form of offsetting the location of the hinge to one side of the joint centerline such that the rigid joint formed between the connecting parts is not primarily dependent on the mating surfaces perpendicular to the long axis of the said elongated members and forming an alternative primary load path between the underside of the first elongated member and the cooperating upper surface of the projecting connector of the second elongated member. In this respect the gap between the elongated members remains constant under normal use and eliminates the possible pinching point when the board is sat or slid on during normal use.

In a preferred embodiment, the elongated members are comprised of shaped plates with stiffeners having a smooth surface on one side for reduce the resistance to sliding while the stiffeners form the joint between the said elongated members on the opposing face.

In a preferred embodiment the said elongated members are provided with folding nibs on the non-sliding side which when opened out form locators on either side of board so as to prevent the board to move laterally when placed over a bath.

In a preferred embodiment the space between the said stiffeners are filled with foam so as to create a series of flat bearing surface against the supporting furniture when the said transfer board is opened for use.

In a preferred embodiment the said elongated members are comprised of metallic component such as Aluminum or Magnesium alloys manufactured by means of casting or assembled from such components.

In yet another embodiment the surfaces used for sliding may be coated by low friction coating such as diamond like carbon.

In a preferred embodiment the said coating forms an elongated strip pattern on the sliding surface.

In yet another preferred embodiment the said strip pattern formed by the low friction coating forms a concentric circle at least on one end of the elongated components.

Embodiments of this invention will now be described by way of example with reference to the accompanying drawings in which:

FIG. 1 Is an Isometric view of the said transfer board when fully opened.

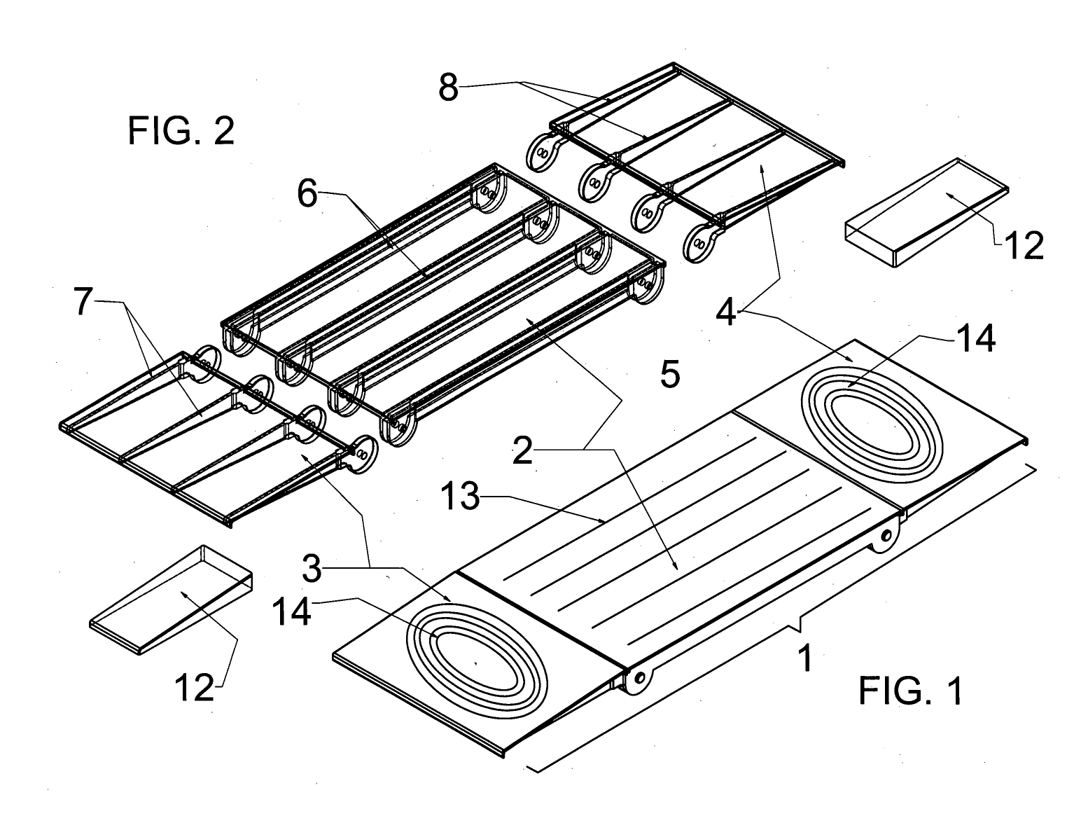

FIG. 2 Is the exploded view of the main components.

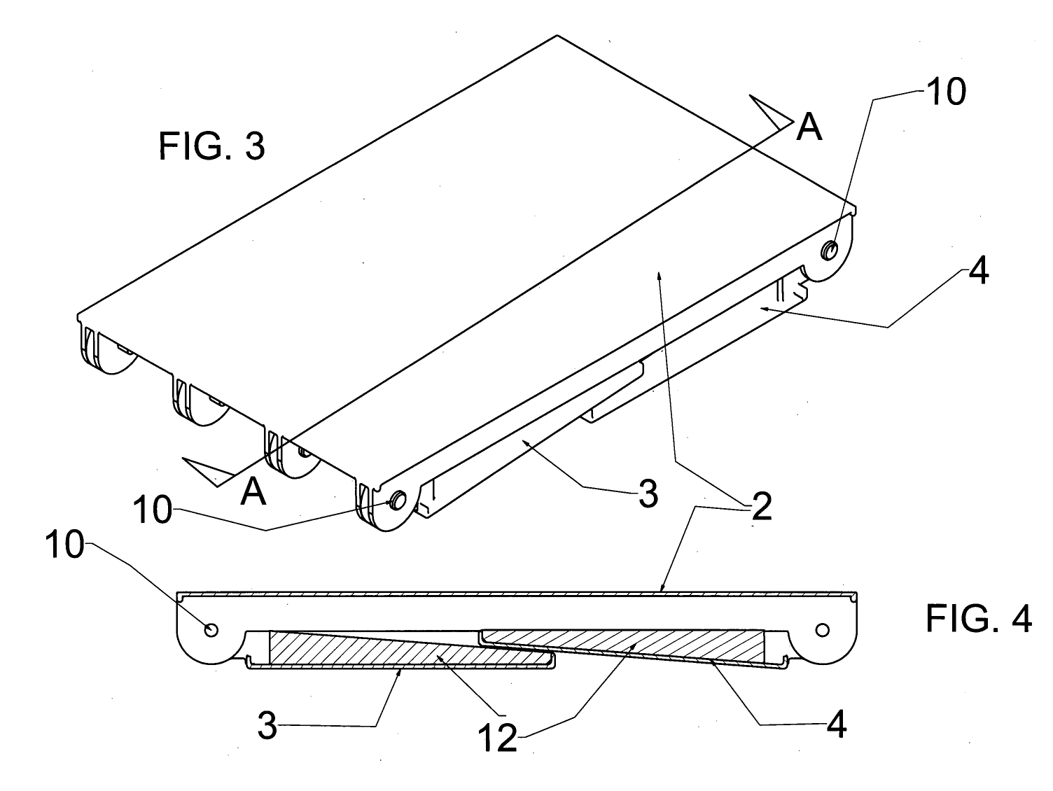

FIG. 3 Is the isometric view of the transfer board when folded.

FIG. 4 Is the sectional elevation along the line A-A as shown in FIG. 1 with the transfer board fully folded.

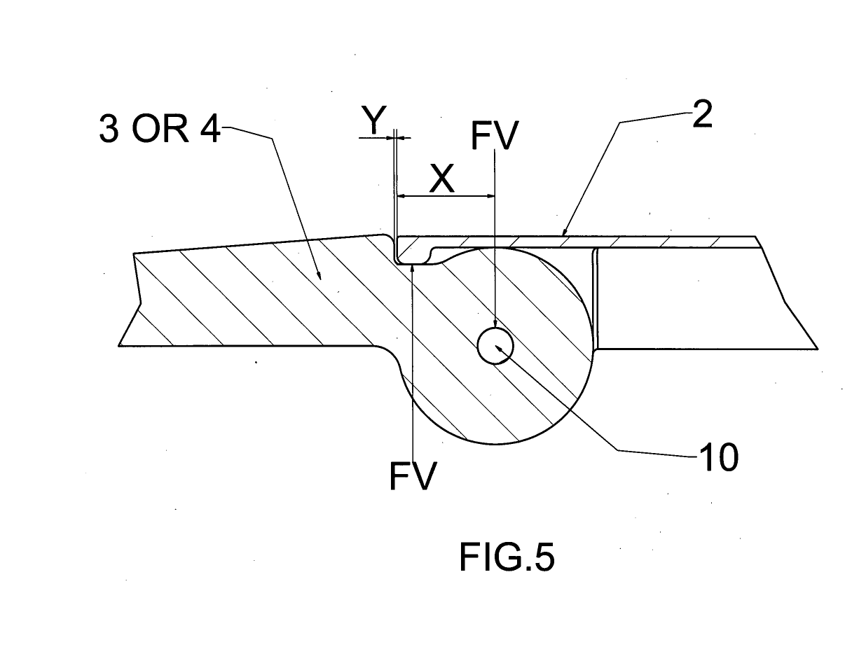

FIG. 5 Is the sectional elevation of the hinge.

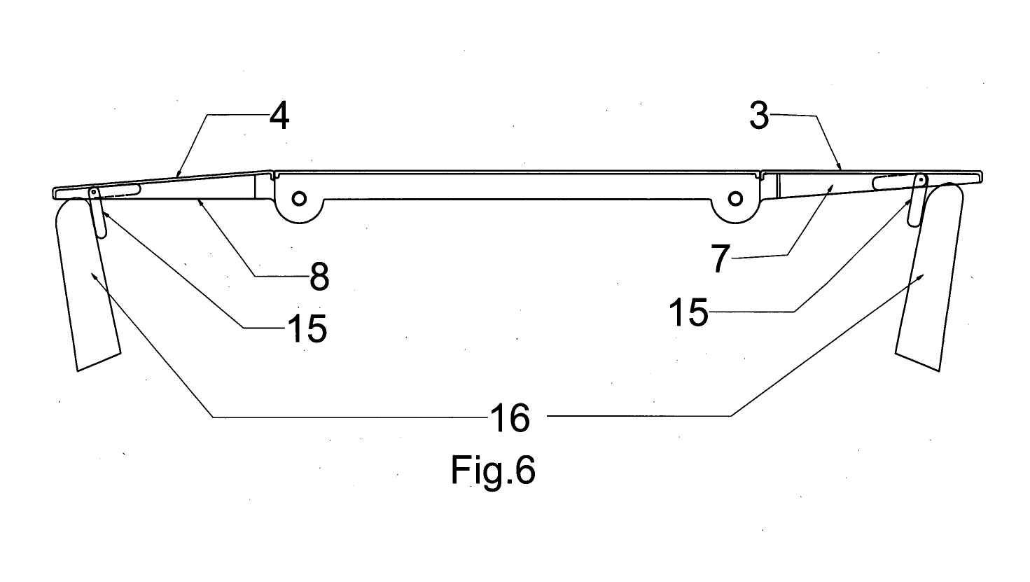

FIG. 6 is the sectional view of the transfer board with locating nibs engaging inside rims of a bath tub transversely.

While the invention is susceptible to various modifications and alternative forms, specific embodiment thereof has been shown by way of examples. It should be understood that the examples and drawings are not intended to limit the invention to the particular form disclosed, but on the contrary, the intention is to cover all modifications, equivalents, and alternatives falling with in the spirit and scope of the invention as defined by the pending claims. Turning to FIGS. 1 and 2, the transfer board (1) constructed in accordance with the present invention, is comprised of elongated members (2,3&4) formed of stiffened plates having stiffeners (6,7&8) respectively and providing a common slide surface (5) when the board is opened for use. The stiffeners (7&8) on the outer elongated members (3&4) projects beyond the plate edge below the underside of the slide surface (5) provided by the centre component (2) and form a hinge joint with its stiffeners (6). Turning to FIGS. 3 and 4 it can be seen that the outer wings of the transfer board can rotate about the hinge pin (10) so as to fold the outer elongated members (3&4) on the center component (2).

The space between the said stiffeners of the elongated members (3&4) may be filled with foam blocks (12) so as to create a flat surface for engaging the furniture surface for support. In a preferred embodiment the said flat surface may be coated by high friction coating to prevent it sliding against the furniture surface. Similarly the space interposed between the stiffeners of elongated member (2) may also filled by foam blocks.

Turning to FIG. (5), the hinge assembly is formed by projecting stiffeners (7&8) of the elongated member (3&4) engaging the stiffeners (6) of the second elongated member (2) via a hinge pin (10). The location of the hinge pin (10) is displaced by a distance (X) from the edge of the elongated member (2) and forms a lever arm between the couples (FV) which resist the moment at the joint, creating a rigid joint when the transfer board is opened for use. The couples FV act perpendicular to the slide surface (5); therefore the gap (Y) between the elongated members does not close, as the primary loading surfaces are provided by the locating pin and the soffit of the elongated member (2), achieving a constant gap at the opposing ends of the elongated members (2, 3&4) at hinge position when the said board is opened for use.

Turning to FIGS. 3 & 4, the elongated members (3&4) can be folded on to the centre elongated member (2) to reduce the length of the transfer board (1) when not in use. The length of the elongated member (2) is such that it allows the wedge shape members (3&4) to fold on one another. Although the representation used a three component transfer board a similar arrangement can be achieved for two part board.

Surface (5) of the transfer board (1) forms the slide surface of the said transfer board (1) and is highly polished so as to reduce the resistance to sliding. In a preferred embodiment the said surface may be coated by low friction coating such as diamond like carbon in the form of longitudinal strips (13) to reduce the friction to sliding longitudinally while preventing the user from falling of the edge transversely.

Similarly the end sections of the said transfer board may be coated by low friction coating forming a concentric circle pattern (14) that allows the user to rotate easily at the ends by reducing the friction when rotating at the end while preventing them from falling of the edge transversely.

Turning to FIG. (6) projecting nibs (15) are hinged between the stiffeners (7&8) of the longitudinal members (3&4). The projecting Nibs (15) can be rotated into a locked position so as to prevent the transfer board from moving laterally when placed over a bath tub by forming shear locating nibs against the bath rims (16).

I claim:

1. A transfer board comprising of at least a pair of elongated members interconnected by means of at least one hinge allowing the elongated members to fold on each other when not in use, wherein the said hinge connecting the said elongated members forms a rigid joint between the said elongated members under the action of load applied to a sliding surface of the transfer board when the said members are unfolded for use; wherein the hinge is offset by a distance ‘X’ from the edge of one elongated member to form bearing surfaces acting perpendicular to the underside of the elongated member while achieving a constant gap between the opposing ends of the elongated members at hinge position when the said board is opened for use.

2. A transfer board as of claim 1 in which the said elongated members are formed of plate members with stiffeners on one face.

3. A transfer board as of claim 1 where in projecting stiffeners of one elongated member engages stiffeners of another elongated member via a pin to form a hinge between the said two elongated members.

4. A transfer board as of claims 1 to 3 wherein spaces interposed between the said stiffeners are filled with foam.

5. A transfer board as of claim 1 where in the sliding surface is covered by low friction coating forming a strip pattern.

6. A transfer board as of claim 5, wherein the said strip patterns are longitudinally arranged in the centre section of the board.

7. A transfer board as of claim 5, wherein the said strip pattern form a concentric circles arrangement at least at one end of the transfer board.

8. A transfer board as of claims 5 to 7 where in the said low friction coating is diamond like carbon.

9. A transfer board as of any of the above claims wherein the said elongated members are of Magnesium or Aluminum alloys.

10. A transfer board substantially as hereinbefore described with reference to the accompanying drawings.
